(19) United States
(12) Patent Application Publication
(10) Pub. No.: US 2009/0123340 A1
(43) Pub. Date: May 14, 2009

(54) WATER QUALITY MONITORING DEVICE AND METHOD

(75) Inventors: N. Eric Knudsen, Maple Valley, WA (US); Jason W. Howell, Maple Valley, WA (US); Renee L. Knudsen, Maple Valley, WA (US)

Correspondence Address: SEED INTELLECTUAL PROPERTY LAW GROUP PLLC, 701 FIFTH AVE, SUITE 5400, SEATTLE, WA, 98104, US

(73) Assignee: H2Observe, LLC, Maple Valley, WA (US)

(21) Appl. No.: 12/245,584

(22) Filed: Oct. 3, 2008

Related U.S. Application Data
(63) Continuation-in-part of application No. 12/113,828, filed on May 1, 2008, now abandoned.
(60) Provisional application No. 60/916,245, filed on May 4, 2007.

(51) Int. Cl. G05B 1/00; G01N 33/00; G06F 15/00; G06F 19/00; G08B 21/00

(52) U.S. Cl. 422/105; 73/61.41; 702/188; 702/25; 340/603

(57) ABSTRACT

A monitor device is attached to the water supply line of a consumer where it repeatedly measures a characteristic that correlates to quality of water in the water line. Each of a succession of a water quality values is derived from one or more of the measurements. The process continues at selected intervals to continuously monitor the condition of water in the line. Each new value can be compared to a reference value representing a maximum acceptable level of contaminants. If the water quality value exceeds the reference value, an overvalue signal is produced, indicating an unacceptable level of contaminants in the water. Additionally, the values can be transmitted to a central collection facility where they are correlated with values transmitted by similar devices on the supply lines of other consumers to track the quality of water of a supply system over time.

This application is a continuation-in-part of U.S. patent application Ser. No. 12/113,828, filed May 1, 2008, now pending, which in turn claims the benefit of provisional application No. 60/916,245, filed May 4, 2007. Both applications are incorporated herein by reference in their entireties.

### BACKGROUND OF THE INVENTION

1. Field of the Invention

The present disclosure relates generally to the field of monitoring water in water lines and collecting associated data, and in particular, to monitoring end-user water supplies to measure characteristics that correlate to water quality or potability, such as dissolved solids.

2. Description of the Related Art

Availability of water is perhaps the most essential factor in determining where humans can live, raise food, and develop industry. Considerable resources are spent finding, moving, storing, and purifying water for human consumption. At the same time, water contamination is a large and growing problem. Point sources, such as sewage systems, feed lots, and industrial waste discharge, combines with non-point sources, such as cropland runoff from fertilizers and insecticides, highway runoff of oil and other automotive products, and atmospheric pollution captured by rain water, to raise the level of contaminants in the water that we rely on. This is in addition to natural mineral contaminants that are collected by water in rivers and aquifers.

Most water contaminants are carried as dissolved or suspended solids in the water. With reference to potable water, purity is commonly classified in relation to the total dissolved solids (TDS) in the water, and is measured in parts-per-million (ppm), by weight. In any given water sample there will be a combination of many contaminants, natural and man-made, that make up the TDS value of the sample. While concentrations of particular contaminants can be harmful at relatively low levels, as a rule, potability is classified in accordance with the TDS value of a water sample. The U.S. Environmental Protection Agency guidelines establish 500 ppm as a maximum acceptable level of TDS for drinking (potable) water. Levels above around 300 ppm usually affect the appearance or taste of water. Tap water in the U.S. generally ranges between 140 and 400 ppm, with water above around 170 ppm classified as hard water. Commercial carbon/particulate filters can produce water in a range of around 50-150 ppm, while water below 50 ppm can be produced by reverse-osmosis filters, distillation, de-ionization, micro-filtration, etc. Extremely pure water below around 25-30 ppm is corrosive to some materials.

Notwithstanding the growing problems and concerns surrounding the question of water purity and availability, most of the water provided in the U.S. for domestic use is well within the standards established, and is safe for use. However, despite this fact, and the fact that most municipal treatment facilities operate to standards that far exceed the minimum standards set federally, most consumers have some level of concern for the quality of their own tap water. These concerns are fueled by news of intentional and accidental spills infiltrating the water supply, and by the aggressive advertising of the growing bottled and filtered water industry. As a result, many people routinely filter their tap water or buy bottled water.

The cost of filtering tap water is generally between three and twenty cents per gallon, while bottled water usually costs more than a $1 per gallon, and can be as high as $4 per gallon. Yet many people are unwilling to trust that their tap water will always be safe, and prefer, instead, to pay a premium for a real or perceived reliable source of clean water.

With regard to surface water, i.e., lakes and rivers, storm run off, industrial effluent, etc., water purity is commonly classified in general terms with reference to TDS and total suspended solids (TSS), which is typically expressed in milligrams per liter (mg/l), and refers to particulate matter that remains in suspension in still water for extended periods without floating or settling. There is a practical upper limit to particle size because larger particles do not generally remain in suspension, but instead either float or settle out. According to one widely accepted definition, TSS refers to particles as small as 0.45 microns. Particulates smaller than this are fall within the dissolved category, and are thus measured as TDS. Measurement of TSS is a rather labor intensive process that is generally performed in a lab using samples obtained in the field, although vehicles can be equipped to carry the necessary equipment for on-site measurement. There is some correlation between TSS and turbidity, which is a measure of the amount of light that is scattered and absorbed by particles in a water sample, and which can be measured using a much simpler operation than TSS. However, the correlation varies according to the composition of the suspended solids, so a measurement of turbidity, for example, cannot be reliably converted to a corresponding value of TSS without first measuring both in a given water sample to establish the correlation. Furthermore, even under ideal conditions the correlation is sometimes only approximate.

### BRIEF SUMMARY OF THE INVENTION

According to an embodiment of the invention, a monitor device is provided that is configured to be coupled to a water supply line and to repeatedly measure a characteristic of water flowing within the water line, and to repeat the process at selected intervals. One example of such a characteristic is electrical conductivity, which is directly related to total dissolved solids in the water, which in turn is correlated to the overall quality or potability of the water. The device is configured to derive a series of water quality values from the measurements. According to one embodiment, each water quality value is compared with a reference value to detect changes in water quality, and provide a response. The water quality value may be, for example, nothing more than a single numerical measurement, or it may be an average of a number of measurements, a comparison of one measurement to a previously obtained measurement, a value representing potability of the water, etc. The response can include outputting an overvalue signal, outputting an audible or visible alarm signal, actuating a water valve, etc.

According to another embodiment, the values are transmitted to a central collection facility where they are correlated with values transmitted by similar devices on the supply lines of other consumers to track the quality of water of a supply system over time. The device can be configured to save the values in a memory and transmit them in packets, or to transmit them as they are obtained.

### BRIEF DESCRIPTION OF THE SEVERAL VIEWS OF THE DRAWING(S)

FIG. 1 is a front perspective view of a water quality monitor device according to an embodiment of the invention.

FIG. 2 is a rear perspective view of the water quality monitor device of FIG. 1.

FIG. 3 illustrates a system for monitoring water quality according to an embodiment of the invention.

FIG. 4 is a flow chart illustrating a method for monitoring water quality in accordance with an embodiment of the invention.

FIG. 5 is a flow chart illustrating a method for monitoring water quality in accordance with another embodiment of the invention.

FIG. 6 is a rear perspective view of a water quality monitor device according to another embodiment of the invention.

FIG. 7 is a side elevation view of the base of the device of FIG. 5.

FIG. 8 is a cross-sectional view of the base of FIG. 6.

FIGS. 9 and 10 illustrate systems for monitoring water quality according to respective embodiments of the invention.

FIG. 11 is a flow chart illustrating a method for monitoring water quality in accordance with another embodiment of the invention.

FIG. 12 shows a system for monitoring and sampling water according to an embodiment.

### DETAILED DESCRIPTION OF THE INVENTION

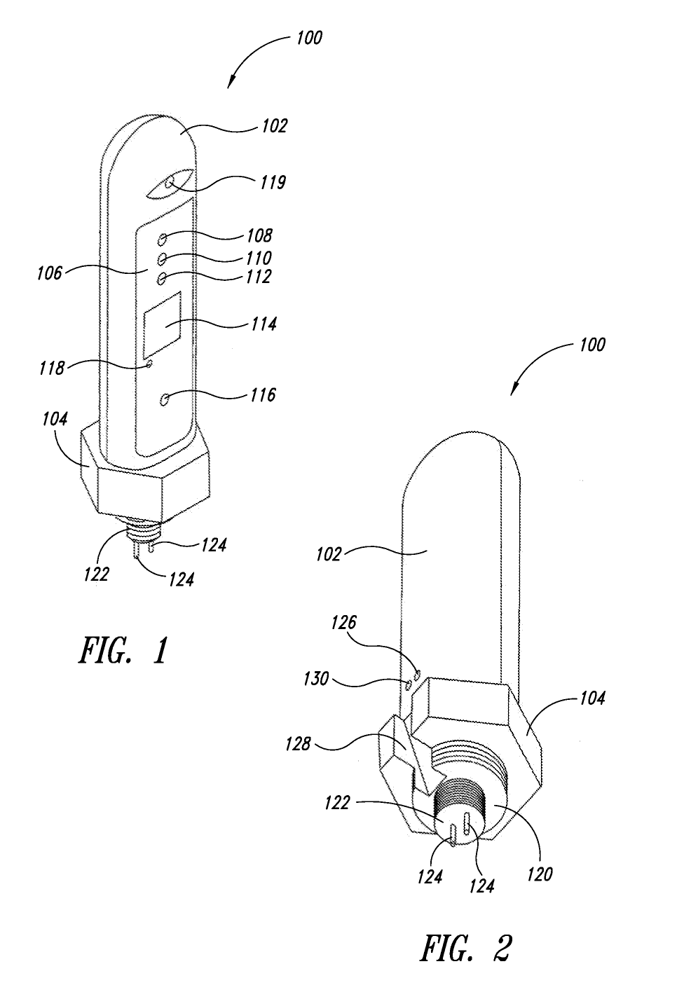

FIGS. 1 and 2 illustrate a water quality monitor device 100 according to an embodiment of the invention. The device includes a body 102 and a base 104. A face panel 106 on a front surface of the body 102 provides access to many of the features of the device 100.

The face panel 106 includes a power button 108, a mode button 110, and a set button 112. A display panel 114, such as, for example, an LCD display, provides information related to user-selected options and water quality measurements. An audio alarm is provided within the body 102, positioned adjacent to an audio output aperture 116. A small reset aperture 118 provides access to a recessed reset button. A status light 119 provides basic operational information.

On a back surface of the body, a power socket 130 is configured to receive a connector from a power cable, while a relay control socket 126 is configured to receive a cable coupling the device 100 to a water shut-off relay.

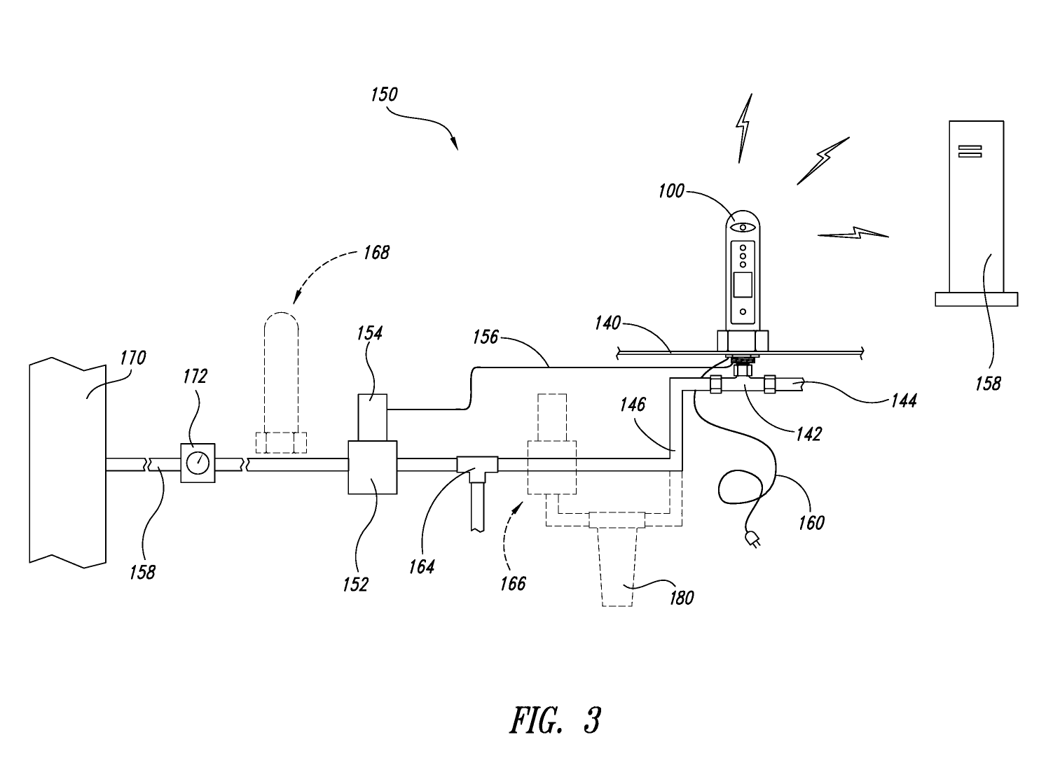

The water quality monitor device 100 of the embodiment illustrated in FIGS. 1 and 2 is configured to be mounted on a sink, as will be described in more detail with respect to FIG. 3. Accordingly, the device 100 is provided with a threaded sink mount 120 extending from the base 104 and configured to be positioned in an aperture formed in the surface of a sink near the faucet. A pipe-threaded water line connector 122 extends below the sink mount 120, and a pair of test electrodes 124 extend from the water line connector 122. A cable bypass channel 128 is formed in a back portion of the base 104 and sink mount 120. The cable bypass channel 128 provides a passage through which the power and relay control cables can pass from beneath the sink surface to couple to their respective sockets 130, 126 in the back of the device 100. Alternatively, sockets or other connection ports are provided for direct access from beneath the sink mount. The monitor device 100 is configured to be mounted such that the electrodes 124 extend into the flow of water in a water line. In operation, the device places a voltage differential across the electrodes 124, thereby causing an electric current to flow in the water between the electrodes 124. The device is configured to measure the flow of electric current and correlate the resulting value with a corresponding level of total dissolved solids (TDS) in the water. This value is displayed in the display panel 114, and updated as the value changes. If TDS rises above a selected threshold, the device 100 is configured to provide an audible alarm, and to provide a control signal for a water control valve, as will be described below.

It is well known that a reliable indicator of the potability of water is the total dissolved solids present in the water. As the level of TDS increases, the suitability of the water for drinking decreases. It is further recognized that there is a high correlation between TDS in water and conductivity. It is possible, therefore, to obtain a relatively accurate measure of the potability of a water sample by measuring the conductivity thereof. The inventors have recognized that it is not necessary to determine specifically what contaminants are present in water in order to determine whether it is fit for drinking. Thus, a nonspecific test such as a conductivity test, which is inexpensive and repeatable, will serve to produce data that a consumer can use to decide whether it is necessary or justified to filter tap water or replace it with bottled water. The inventors have devised the water quality monitor configured to repeatedly perform water conductivity tests at selected intervals to monitor water quality in a location such as, for example, a residence.

Referring now to FIG. 3, a monitoring system 150 according to an embodiment incorporating the water quality monitor device 100 of FIGS. 1 and 2 is described. The monitoring system 150 will be described with reference to a residence as the water consumer. However, the system may be employed anywhere water quality is a concern. In particular, businesses that require potable water can benefit from such a system. These may include, for example, restaurants, food processors, bottling companies, etc. Accordingly, the scope of the invention extends to include such water consumers, as well.

The monitoring device 100 is mounted to an upper surface of a sink 140 with the water line connector 122 extending below the sink 140. A tee junction 142 is threaded onto the water line connector 122 such that the electrodes 124 extend into the stream of water flowing through the junction 142. A water supply line 146 (usually the cold water supply line) is connected to one end of the tee junction 142, and a continuation 144 of the water supply line 146 extends between the tee junction 142 and the faucet of the sink 140.

An optional control valve 152 may be positioned between a feeder line 158 from the municipal water main 170 and a first branch 164 of the water line within the residence. A valve relay 154 is electrically coupled to the control valve 152 to control operation of the valve. The relay 154 is coupled, via a relay control cable, 156, to the water quality monitor device 100. The relay control cable 156 traverses the cable bypass channel 128 to access the relay control socket 126 of the device 100.

A power cable 160 also traverses the bypass channel 128 to provide power to the device 100 from a power source below the sink. Most kitchen sinks include access to electrical power beneath the sink to provide power for dishwashing machines and waste disposal units. Accordingly, the DC power cable 160 for the monitoring device 100 is easily connected to power, either via a standard power outlet or by direct wiring.

The water quality monitor device 100 is configured to repeatedly monitor the TDS of water passing between the electrodes 124. In the event the level of solids in the water rises above the selected threshold, the device sounds an audible alarm and signals the relay 154 to energize the valve 152 to shut off the water. Thus, the user can use water directly from the municipal supply with confidence that the water does not contain TDS above the selected threshold.

According to an alternate configuration to that described above, the control valve 152 may be positioned downstream from the branches that supply water to outside hose bibs, irrigation, and even laundry and bathrooms, as indicated in dashed lines at 166 in FIG. 3. In this arrangement only drinking water is shut off when the valve 152 operates. Such a configuration allows a residence to continue using untreated water for non-potable applications even when incoming water is not suitable for drinking.

According to another configuration, with the control valve in the position 166, the monitor device 100 is positioned upstream from the first branch 164, as shown in dashed lines at 168, while the control valve 152 is positioned downstream from the monitor device in the branch that supplies drinking water, as shown at position 166. Thus, only the drinking water is shut off, while water continues to flow past the monitor device 100 regardless of the valve setting, so that when the incoming water is again safe to drink, the device 100 will detect the drop in TDS and restore the valve 152 to its open setting. Furthermore, according to an alternative embodiment, a permanently installed filter 180 is connected to the system via the valve 152, which is configured as a two-way valve rather than an on-off valve. When an overvalue signal is provided by the monitor 100, the valve 152 routes at least the potable water through the filter 180 to provide a continuing supply of water until the water quality is restored to an acceptable level. In this way, a supply of safe drinking water is ensured, while the expense of unnecessary filtering is avoided.

According to an embodiment of the invention, a separate power relay device is provided (not shown), to which the relay control cable 156 is coupled. This relay device is configured to provide power to an accessory such as valve 152, and may also, or alternatively, be coupled to control other functions.

The water quality monitor 100 of FIGS. 1-3 has been illustrated and described for use on a sink or counter top. In this location, a user has easy access to the device to confirm that the device is operative and to note the water quality level, as indicated on the display panel 114, at any time. However, it will be recognized that the device 100 may be installed on a water line at any convenient location, and that it is not essential that the device 100 be instantly accessible.

According to an alternative embodiment, a monitor is provided that is configured to be positioned elsewhere along an incoming water line. For example, the monitor may be coupled to the feeder line of the residence in a location such as at the municipal water meter 172, an underground utility box, a garage, or crawl space. Furthermore, the monitor may be configured to include remote location of control and display functions such that a user can review and control operation of the device without actually going to where the sensor portion of the device is positioned. For example, a wall mounted panel may be provided for this purpose. Alternatively, appropriate software and hardware modules can be provided such that the user can control and check the monitoring device from a computer.

According to other embodiments of the invention, a monitor is provided for use in vehicles that have potable water supplies, such as, for example, recreation vehicles, motor coaches, boats, aircraft, etc. The monitor may be provided as an after-market device, or it may be an integral part of the originally installed water system. Some components of the monitor may be coupled to a water supply line of the vehicle, while other components are provided in a control panel located elsewhere in the vehicle, or may be integrated with other instrumentation of the vehicle so as to be incorporated into the vehicle's overall control scheme. Such alternate configurations are within the abilities of one of ordinary skill, and fall within the scope of the invention.

While the monitor device of the embodiment described with reference to FIGS. 1 and 2 provides an overvalue alarm when TDS exceeds a single selected threshold, the device obtains continuous readings of the actual level of TDS. So, for example, if a transitory rise in TDS occurs that does not reach the threshold, it will go unnoticed, unless a user is actually watching the display panel at the time. Accordingly, embodiments are provided in which data collected by the monitor device 100 is stored in an internal memory for later retrieval, or transmitted to a separate storage device such as a computer or other device capable of accepting and storing data. In the embodiment illustrated in FIG. 3, the data is transmitted wirelessly to a nearby computer CPU 158. Alternatively, the data may be transmitted to the separate storage device via wired connection or other transmission means, such as, for example, HomePlug™, Homepna™, or ZigBee™.

By storing the data obtained, a user can track changes in water quality over time, in order to obtain a long-term water quality image. Data from the continually operating device is stored for later review. Thus, a user can note conditions that would otherwise be un-noticed. For example, the water at a given location may be constantly high in TDS, though below the threshold; or there may be frequent spikes in TDS that approach but don't reach the threshold. In another case, the water quality may vary by season, or after heavy rain. These or other conditions may prompt the user to adjust the threshold of the device if they feel that adjustment would be acceptable or take steps to obtain filtered water at those particular times.

According to an embodiment, a user programs the operational parameters of the device by selecting and entering desired settings at a personal computer, then uploads the instructions to the monitor device via a wireless communication link. According to another, the monitor device transmits readings to the computer, which then performs some or all of the remaining processing steps.

According to an embodiment of the invention, the data is transmitted from the monitor or storage device to a central collection facility, where it is compiled with similar data from other residences and businesses. This data may have particular benefit when compiled and compared with data from other monitors around a given region. For example, the local storage device may be programmed to periodically transmit data via an internet connection, or some other transmission link. Alternatively, the monitor device itself may be configured to transmit data to a collection network. Homeplug™ and ZigBee™ are examples of protocols that are known in the art and can be employed for this purpose.

Centrally collecting such data allows municipal authorities to detect and track problems such as aging or damaged pipes, contamination in watersheds, etc., and predict upcoming problems based on historic trends. Limited financial resources for upgrading water supply systems can then be allocated in a way that provides maximum benefit. National governmental agencies can use the data to locate or track widespread or regional problems such as low-level contamination of river systems, groundwater, or aquifers, that can arise, for example, from industrial, agricultural, or residential pollution, to properly allocate funding for treatment and enforcement, and evaluate progress of such programs. There will still be a need for on-site water testing for evaluation of specific contaminants or events, but, in accordance with the principles of the invention, first order tracking can be done more timely, economically, and comprehensively than is currently possible.

Additionally, there are commercial benefits to collection of such data locally and nationally. For example, the provision of clean water can be a significant expense to a food processing company, especially if the water must be retreated in the plant before use. Thus, data that can show water quality trends geographically and over time can be very useful when a location is being sought for a processing facility. Such data may also be of use to companies that provide products designed to address water quality issues in homes or businesses, by allowing them to target specific areas that have a higher need of their products.

Furthermore, even where a particular water consumer is not concerned with the potability of its water, other parties may have an interest in comprehensive water quality data, and to that end they may wish to obtain readings from many or all water consumers that fall within a given category. Water consumers can be categorized by, for example, region, volume of consumption, water source, age of connecting lines, distance from source, intended use of water, time of peak use, etc.

Thus, a municipal water authority, for example, may wish to emplace monitors with selected consumers at its own expense, or it may provide incentives to those consumers to obtain monitor devices, thereby sharing the expense. The water authority obtains comprehensive data regarding water quality within the selected category or categories of consumers, while the individual consumer obtains data that is specific to the water it uses.

According to an embodiment, a remote sensor device is configured simply to take regular water quality readings and transmit them to a receiver such as, for example, a central collection facility, either wirelessly or by physical connection. The transmission may be continuous, periodic, or intermittent. The device can be configured to obtain readings only when transmitting, or to store data between transmissions. The device can also be configured to obtain one or more readings only when it is prompted, such as when the receiver indicates it is prepared to accept a transmission. As noted above, the receiver may be a central collection facility, or it may be an intermediate collection facility such as a relay, a facility dedicated to the one or more devices and configured to collect and forward data, or a mobile collection facility that periodically connects to receive data, either wirelessly or by physical connection while nearby, then moves to another location.

The sensor device can comprise more than one discrete component. For example, the device can comprise a first component that houses test electrodes and that is configured to be coupled to the consumer's water line, while other elements, such as power supply, transmitter, receiver, storage, control circuitry, etc., are housed in a separate component or components.

The sensor device can be configured to provide a unique identifying code so transmitted data can be associated with specific devices. Alternatively, the device can be configured to transmit at a specific time, and the receiver is configured to identify the source of data according to the transmission time. According to another embodiment, the device is configured to transmit upon detection of a specific signal or code unique to that device, or at least unique within a defined area. To collect data, the receiver transmits the specific signal, whereupon the sensor device responds by transmitting data. Thus, the specific device is identified to the receiver by virtue of having responded to the unique signal.

According to an embodiment, the sensor device is configured to obtain water quality readings and transmit the readings together with an electronic time-stamp indicating the time the reading was obtained. When the data is collected from a plurality of such sensor devices, and compiled, an overall image of the water supply network can be created, showing fluctuations of water quality over time and across the system. In one embodiment, the device is configured to store the data for extended periods of time, then transmit in data packets when pinged by a receiver. Alternatively, regional relay receivers are positioned so that each sensor device is within range of at least one such receiver, and can therefore transmit substantially in real time, rather than storing the data for later transmission. According to a further embodiment, cellular capable monitors are placed with specific consumers to create a mesh network, and an appropriate networking system, such as, for example, Zigbee™ is employed to collect data from the surrounding monitors for data transfer through the cellular capable monitors.

According to an embodiment, the device is configured to encrypt the data prior to transmission. This may be of use in cases where the data has commercial value, or includes proprietary information, or where the privacy of the consumer is a concern.

Where the sensor device is powered by a battery, the frequency of the readings can be selected to extend the battery life. According to another embodiment, the sensor device collects a very small amount of power from the passage of water to power its operation, storing the collected power in a capacitor or battery, and drawing a portion of the stored power to take readings and transmit data. According to a third embodiment, a solar collector is provided to power the sensor device. The collector can be integrated, for example, into the lid of an underground utility box.

Preferably, where the sensor device is one of a plurality of similar devices, each positioned at or near a respective consumer's water meter. Where the party providing and installing the sensor device is the local water authority, the device can be installed at the consumer's water meter or upstream therefrom in the feeder line extending from the water main to the water meter. Alternatively, the sensor device can be integrated with the meter as a single unit. Ideally, the sensor device is positioned immediately downstream from the valve portion of the water meter so that the water can be shut off at the valve to install and service the device.

The data collected from this location will not be affected by the material, quality, or age of pipes belonging to the consumer, so the party collecting the data can obtain more accurate information in a more controlled fashion regarding the quality of water delivered to the consumer. In contrast, a sensor device that is positioned close to where the water is used, such as near a faucet or machine intake, for example, may be affected by attributes of the consumer's water pipes, which reduces the accuracy of the data provided as it relates to the municipality's system and additionally as it compares to neighboring properties in a geographic region, due to the lack of control of the respective users piping attributes. For example, if the consumer's water pipes are excessively corroded or rusty, or have significant mineral layers, or contain elements that leach into the water, such conditions can affect the readings of the sensor device. The potential for such effects reduces the cross comparibility of the data collected. Nevertheless, even where the devices are not optimally positioned, the data can still provide valuable information to the party collecting the data. The average condition of the water lines of a given category of consumer can be determined, by representative sampling, for example, and compensated for in the collected data to provide usable information regarding the distribution network.

In some cases, a consumer may wish to obtain one or more separate secondary sensors to be positioned closer to the points of use. This may be especially valuable in large facilities such as schools and campuses, factories, and office buildings. By employing multiple sensor devices near the end use points of the multiple lines within the facility, detailed information can be obtained and very localized problems or contamination detected. Hypothetically, for example, some pipes of a facility may have been recently replaced, others may still be in good condition, while yet others may be leaching high concentrations of lead or other potentially adverse elements into the water. A network of sensor devices within the facility can assist in detecting pipes that require immediate replacement and avoiding unnecessary replacement of sound pipes.

Alternatively, a portable sensor unit can be used to obtain a reading of water at the consumer's point or points of use and the reading compared to a reading obtained by the sensor device located at the consumer's water meter, in order to detect problems in the consumer's water lines. Such comparison readings may only be necessary at long intervals, as the condition of the water lines will not vary over a short period to a significant degree. The portable sensor device may be made available by the local water authority, or rented by the consumer.

In addition to the basic sensor device, which may be provided and installed by a municipal water authority, according to an embodiment, additional components are provided for the use of individual water consumers. The components are configured to operate with a sensor device coupled to a consumer's water supply and provide specific benefits to the consumer. According to an embodiment, a display component is provided, configured to receive and show data collected by the sensor device. The display component can include a memory configured to store the data for use in tracking changes over time. The display component can also be configured to perform threshold comparisons or other operations, to transmit commands to valves, to emit alarm signals, etc., similar to functions disclosed with reference to monitor devices of other embodiments.

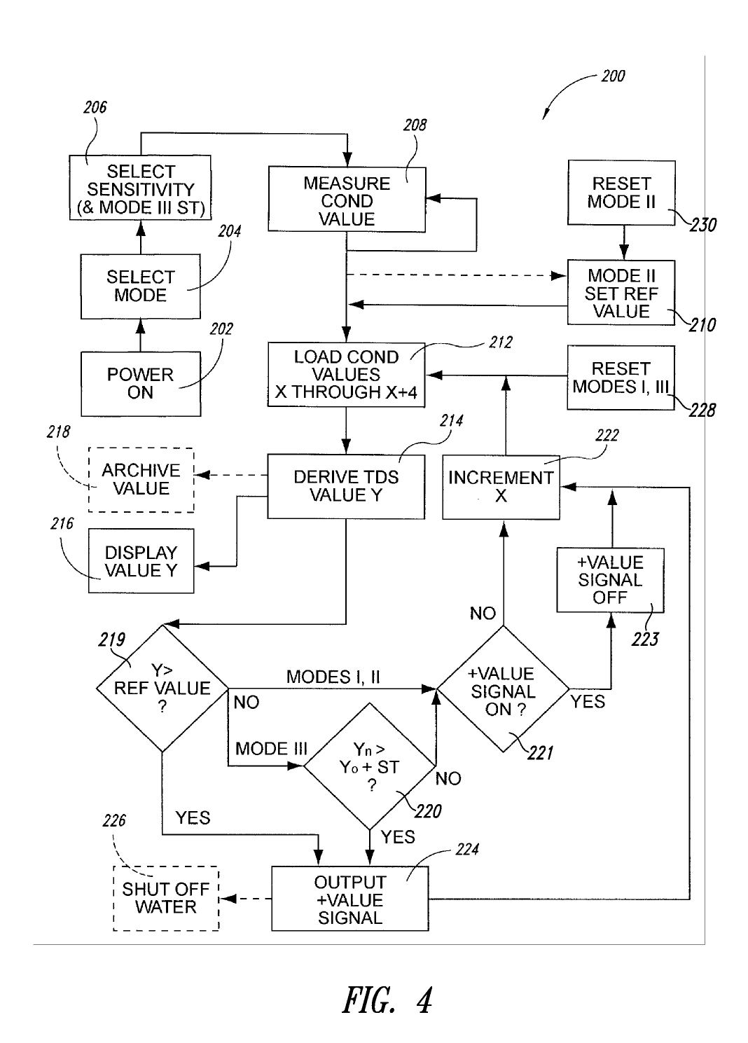

Referring now to FIG. 4, a flow chart 200 is provided, which outlines operation of a water quality monitor according to an embodiment of the invention. Following a review of the steps outlined in FIG. 4, the operation will be described in more detail in relation to the monitor device 100 of FIGS. 1-3. It will be recognized that other monitor devices can also be configured to operate in accordance with the operation outlined here.

The process begins at step 202 with turning on the power to the monitor. At this point in step 203, the monitor initiates by obtaining the measured conductivity values of thirty consecutive readings and computes an average of those readings, compares that average to a table of correlating TDS values, and establishes the initialization value (Init) for further comparison until the system is reset or powered down and rebooted. At this point, the monitor simultaneously sets a mode II reference value based on the factory setting for mode II and “Init” and begins performing conductivity tests, or readings, at timed intervals (step 208), such as, for example, one second intervals, and repeats the tests continuously until the system is powered down. In one embodiment, the user then selects a mode of operation for the device, step 204, and a level of sensitivity is selected by the user in step 206. The user may also select a slope threshold at step 206, as will be described in more detail later, in the discussion of mode III operation. If mode II is selected at step 204, the user has the option to accept the factory setting for mode II or change the setting at step 206. The system sets a mode II reference value at step 210 during the first cycle of the process. If either of modes I or III is selected, and after the first cycle of mode II, step 210 is omitted. At 212, the system obtains the measured conductivity values of five consecutive readings obtained in the measurement step 208. The value X of step 212 refers to the first, in a continuous sequence of measurements, of five consecutive readings. When X is incremented, as discussed below, this does not mean that a number value in a buffer or memory is necessarily incremented, but that the second of the five consecutive readings becomes the first of a new set of five readings.

At step 214, an average of the five readings is obtained, which is correlated with the TDS corresponding to that value of conductivity to produce a TDS value Y. The value Y of TDS is then displayed at panel 114 of the device (216) until it is replaced by another value. Optionally, at step 218, the system also transmits the value Y to a storage medium or remote storage facility.

Simultaneously with the display step 216, the TDS value Y is compared, in step 219, with the reference value previously established. If the value Y does not exceed the reference value (the NO path), the system proceeds either to step 220, if in mode III, or step 221, if in modes I or II. Step 220 is a slope threshold comparison, which will be discussed in more detail below. At step 221, the system checks for an existing overvalue signal and, if no signal is detected, the value of X is incremented at 222, and the system returns to step 212. If a preexisting overvalue signal is detected at step 221, the signal is terminated at step 223 and the system proceeds to increment X at step 222. By incrementing X, the first of the original five measured conductivity values is discarded, and a new fifth value is obtained for the averaging step 214. This is sometimes referred to as a moving window sampling method.

If the value Y exceeds the reference value at step 219, the system outputs an overvalue signal at step 224 by displaying an overvalue alert on the display panel. This signal may also include an audible alarm, flashing of a status light, or any other appropriate signal. At this point, the system may optionally send a control signal to change the setting of a water valve, at step 226. The overvalue signal continues while the system increments the value of X at 222, and the system returns to step 212. When the overvalue signal is produced, the user can silence an audible alarm without otherwise changing operation, reset the system (steps 228, 230), or power down the system. If the water quality value returns to an acceptable level before user interruption, the overvalue signal is ended during the next cycle, at step 223.

A reset in modes I or III (step 228) will restart operation of the system at step 212, where five new values are loaded and the process continues. A reset in mode II, at step 230, also resets the mode II reference value.

Referring now to the operation of the water quality monitor 100, step 202 of the process outlined in FIG. 4 is performed by pressing the power button 108. According to one embodiment, when the power button is pressed for less than three seconds (normal start), the system turns on and performs a self check, then waits for the user to select mode and sensitivity. When the power button is pressed for more than three seconds (fast start), the system turns on, performs a self check, and begins operation based on the most recent mode and sensitivity settings provided. If the power button is pressed for less than three seconds while the system is running, a back light behind the display panel lights up temporarily (e.g., seven seconds), then goes out. If the power button is pressed for more than three seconds while the system is running, the system shuts down.

In step 204, the user selects a mode of operation by pressing the mode button 110. Repeatedly pressing the mode button 110 cycles through the available modes of operation, which are displayed on the display panel 114 as the modes are selected. According to one embodiment, there are three modes of operation: absolute measurement (mode I), relative measurement (mode II), and slope measurement (Mode III). When the absolute measurement mode (mode I) is selected in step 204, the user selects a maximum acceptable value of total dissolved solids in step 206, beyond which the system will signal an overvalue. The system is provided with a default value, such as, for example, 400 parts-per-million (ppm), but the user has the option to change this value.

For example, in one embodiment, the user can select values ranging between 10 and 1500 ppm. The value is selected by repeatedly pressing the set button 112 of the monitor device 100. The selected values cycle to progressively lower threshold values, in increments of 10 ppm at values below, for example, 100 ppm, i.e., 90, 80, 70, down to the minimum value, then cycling to the maximum value and continuing down from the maximum value in increments of 100 ppm at higher values, i.e., 1500, 1400, 1300, etc. The selected values are shown in the display panel as the user cycles through. When no change is made for a selected period, i.e., seven seconds, for example, the displayed value is set as the reference value. In another embodiment, the user enters a key sequence to set the value.

When mode I is selected in step 204, the reference value selected in step 206 is used in the comparison step 219. When TDS, as reflected by the average value obtained in step 214, exceeds the selected reference value, the process proceeds to step 224 where the overvalue signal is produced. The overvalue signal continues as long as Y exceeds the reference value in step 219. The process increments X at step 222. If the overvalue signal is in response to a temporary spike in TDS, after which the TDS level drops below the threshold value, the system terminates the overvalue signal at step 223 and returns to normal operation, once the TDS level drops below the threshold value. However, if the signal is due to an overall rise in TDS, the system continues to produce the overvalue signal. The user can change the selected sensitivity setting at 206 to prevent the system from producing the overvalue signal, can silence an audible alarm while permitting the system to continue to monitor TDS, or can change the system to another mode of operation.

If mode II, relative measurement mode, is selected in step 204, the system uses, as a threshold value, a number that is established relative to a measured value. When the system is reset or powered up, the system provides a default multiplier, such as, for example 2.50, though the user can change the multiplier in step 206. In one embodiment this is accomplished by repeatedly pressing the set button 112 until the desired multiplier is displayed in the display panel. In one embodiment, the user can select multiplier values of between 1.10 and 4.00, in increments of 0.10. Additionally, the increments may be spaced further apart at the upper end. Once a multiplier value is selected, the system proceeds to step 208, and begins obtaining readings, and moves to step 210, where a reference value is set. To set the reference value, the system obtains a number of consecutive readings, such as, for example, thirty readings obtained at one second intervals. An average value of these readings is then derived and that value is multiplied by the multiplier selected in step 206. The resulting figure is saved as the reference value for the comparison step 220. The system then continues operation as previously described. If an overvalue is detected (224), the overvalue signal is produced (228) until the system is reset, powered down, or returns to a undervalue condition. When the system is reset in mode II (230), a new reference value is obtained and operation proceeds, with a new reference value.

Mode III is a slope measurement mode. Slope refers to a degree of change over time, so a slope threshold value is the maximum acceptable change of Y within a given time period. If mode III is selected at step 204, the user also selects a slope threshold ST at step 206, or confirms a preset threshold. In other respects, the system operates according to mode I (absolute measurement), except that the system is also sensitive to sudden changes of TDS, even if the maximum TDS does not exceed the selected mode I threshold. For example, if the user selects mode III, the system operates under the most recent settings established under mode I, but the system also holds each derived value Y (214) for a selected period of time such as, for example, 30 seconds, and, in addition to comparing the newest value Y with the threshold value (220), at step 234 it also compares the newest value Y with a value Y saved 30 seconds previously. If a difference between the two derived values exceeds the selected mode III slope threshold ST, the overvalue signal is produced.

Thus, for example, if the selected mode III slope threshold ST is 120, the mode I threshold value is 400 ppm, and the previously saved value Y is 100 ppm, the system will signal an overvalue if the new derived value Y is greater than 220 ppm, indicating that TDS has increased more than 120 ppm over thirty seconds. On the other hand, a more gradual rise will not trigger the overvalue signal until the level reaches 400 ppm, at which time the system will respond as described with reference to mode I operation. The system provides a default mode III threshold value, but the user can change the slope threshold ST at step 206 as described with reference to modes I and II. The mode and sensitivity settings can be set at any time by restarting the system.

According to another embodiment of the invention, the system is configured to operate under modes I, II, and III simultaneously, such that an overvalue signal is produced if any of three thresholds are exceeded. The device includes default values for each threshold, which a user can change as described above.

Combinations of color and blink patterns of the status light 119 may be used to signal various conditions of operation. For example, a steady green light indicates normal operation of the device, while a steady red light indicates an overvalue signal. Flashing red may be used to indicate a power failure and operation on back-up battery power. These and other conditions may also be indicated in the display panel.

By correlating five readings to arrive at a TDS value, false alarms are minimized. For example, it is not uncommon for a fleck of metal or rust to separate from an inside wall of a water line and travel with the water down the line. Such a particle would induce a brief spike in the conductivity reading as it passed between the electrodes of the device. Obtaining an average of a plurality of readings helps to prevent such a spike from provoking an overvalue signal. It will be recognized that the number of readings that are averaged to obtain the TDS value is a design choice, and is not limited to the five readings described with reference to FIG. 4.

The term average, as used in the specification and claims, is intended to refer to any process of data manipulation to extract a single value from a number of different readings. This value may be derived by any appropriate method, including, for example, calculation by arithmetic mean, median, or mode. Furthermore, circuitry, such as integrating or differentiating circuits, for example, may be employed to produce similar results without calculation.

In step 208, the readings may be obtained continuously or at any convenient interval, such as one second as described, or more or less frequently. For example, readings may be obtained at intervals of ten seconds, one minute, or many minutes. The selection of the interval length is a design choice that will be dictated, in part, on factors such as the volume of water flowing in the water line, whether flow is continuous or intermittent, availability of power, etc. Typically, the interval is set in the factory, but the device can be configured to permit the user to select or change the interval. In the case of a constant reading, i.e., a constant current between the device electrodes, circuitry or software of the device may be configured to obtain the necessary information from the continuous flow of data.

According to some embodiments, the system reads TDS at spaced intervals, of for example, once every five or ten minutes. According to another embodiment, the system operates in response to specific criteria. For example, the system can include a sensor configured to detect water flow, and to operate only while water is moving in the water line. This prevents the system from sampling the same water repeatedly while downstream faucets are closed. According to another embodiment, the system operates at a rate that is proportionate to the rate of flow of water in the line, such that, while there is no flow, the system takes no readings, and as flow increases, the sample rate increases proportionately. This may provide more accurate data regarding overall quality of water in the line. According to yet another embodiment, the system obtains a sample reading only when a command is provided, e.g., when a remote device pings the system or when connection with the remote device is achieved, to obtain a reading or readings.

As described above, according to some embodiments, a number of readings are averaged to prevent stray spikes from producing false signals. Other methods can also be used, such as, for example, limiting the amplitude of change from one reading to the next, or ignoring readings that exceed an allowed amplitude of change. Thus, transitory changes will tend to be ignored, while persistent changes will result in step-wise increases until a threshold is reached.

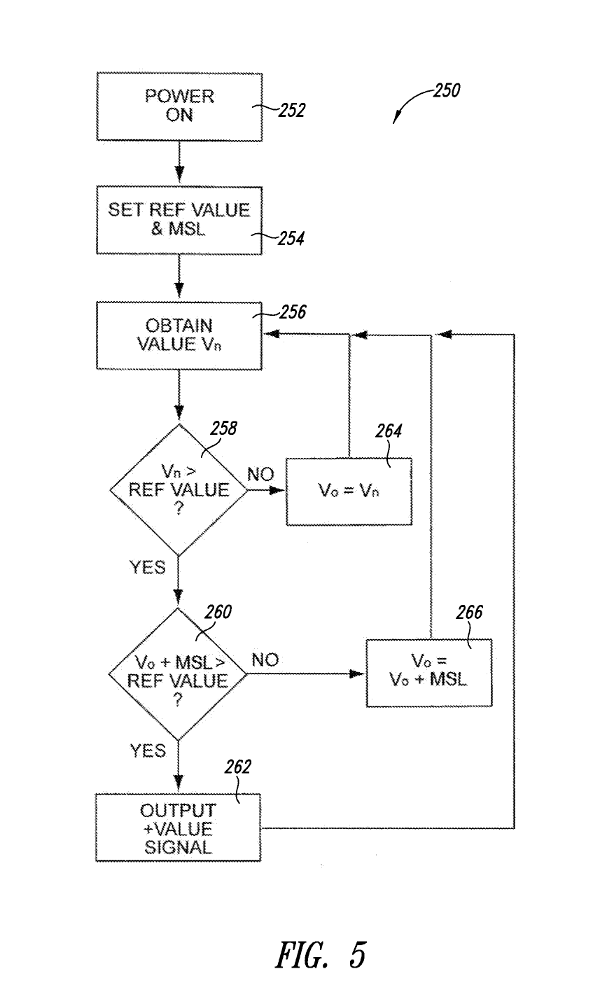

A flow chart 250 illustrating one such method is shown in FIG. 5. According to the disclosed embodiment, after powering up the system (202), a reference value and a maximum slope limit (MSL) are selected (254). A new water quality value Vₙ is obtained (256) and compared with the reference value (258). If the new value Vₙ is greater than the reference value, the most recent water quality value previously obtained, i.e., the old water quality value Vₒ, is compared to the reference value (260). If the difference between the old value Vₒ and the reference value is less than the maximum slope limit MSL, an overvalue signal is produced (262) and the process returns to step 256, and repeats.

If, at step 258, the new value Vₙ is less than the reference value, the old water quality value Vₒ is discarded and the new water quality value Vₙ is saved as the old water quality value Vₒ (264) and the process returns to step 256, and repeats. If, at step 260, the difference between the old value Vₒ and the reference value is less than the maximum slope limit MSL, the old water quality value Vₒ is increased by an amount equal to the maximum slope limit MSL (266) and the process returns to step 256, and repeats.

As can be seen, in this embodiment, the value that ultimately triggers the overvalue signal can only increase in increments established by the maximum slope limit. Thus, transitory spikes in the water quality characteristic being monitored will generally be ignored, while more persistent increases will be detected after a few cycles of the system. On the other hand, in systems that are configured to save data, the actual readings can be preserved by saving each reading as it is obtained.

It should be noted that in the embodiment disclosed above, the water quality value is a measure of an inherent water characteristic, such as, for example, TDS, that is inversely related to the actual purity or potability of the water, so that an increase in value indicates a corresponding decrease in purity. In a system that is configured to measure a characteristic that is directly related to purity or potability, so that an increase in value indicates an increase in purity, operation of the process described is substantially inverted. The reference value becomes a minimum acceptable value, and the maximum slope limit acts to limit the maximum permissible drop in value for a given process cycle.

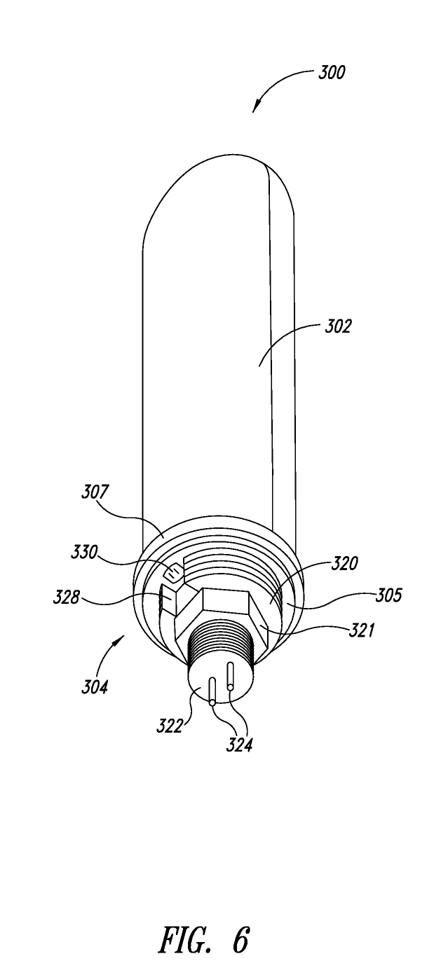

Referring now to FIGS. 6-9, a water quality monitor device 300 according to another embodiment of the invention will be described. The device of FIGS. 6-9 differs from previously described devices primarily with respect to the base 304 and associated elements by which the device is mounted to a sink.

The base 304 includes a rim 307 and a projection 305 extending below the rim. A threaded sink mount 320 extends downward from the base and a threaded water line connector 322 extends below the threaded sink mount 320, with test electrodes 324 extending therebelow as with previous embodiments. Between the threaded sink mount 320 and the threaded water line connector 322, a wrench contact 321 is provided. In the pictured embodiment, the wrench contact 321 is hexagonal, for engagement by any of a number of common wrenches to aid in installation. The wrench contact 321 may, alternatively, have some other configuration appropriate for a specialized tool. A cable bypass channel 328 is provided for access, from below a mounting substrate 340, to a power socket 330 and other associated connections such as have been described previously. In the embodiment of FIGS. 6-8, the cable bypass channel 328 extends from below the threaded sink mount 320 upward to terminate at a bottom surface of the base 304.

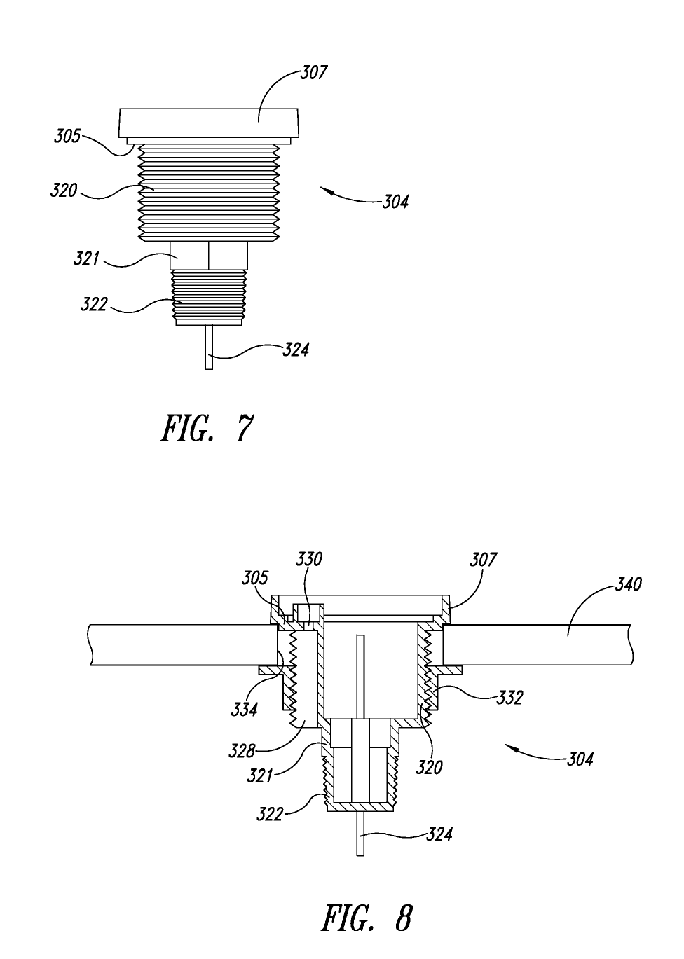

Referring in particular to FIG. 8, a cross-section of a lower portion of the device 300 is shown mounted to an appropriate mounting substrate 340 such as, for example, a sink or counter surface. An opening 334 is provided in the mounting substrate 340 sized to be slightly larger than a size of the projection 305. When the device 300 is properly positioned on the mounting substrate 340, the projection 305 extends some distance into the opening 334, while the rim 307 rests on an upper surface of the mounting substrate 340. The upper portion of the base 304, including the rim 307, can have any selected shape such as, for example, circular, oval, square, etc. The projection 305 can be circular in shape for installation into a circular opening on the mounting substrate. However, provided that the dimensions of the projection 305 are such as to fit within the opening 334, and the dimensions of the rim 307 are such as to extend beyond the opening 334 to rest on the upper surface of the mounting substrate 340, the respective shapes of the rim 307, the projection 305, and the opening 334, are generally not critical. According to one embodiment, the opening 334 and the projection 305 each have a shape that is non-circular, such as, for example, oval or elliptical. With the projection 305 engaged in the opening 334, the base 304 cannot rotate on the substrate 340, meaning that it is not necessary to tightly hold the body 302 or base 304 during installation.

According to an embodiment, a gasket or other appropriate sealing means (not shown) is provided between the rim 307 and the upper surface of the mounting substrate 340, so as to provide a moisture-proof barrier to prevent liquids that might at times be present on the surface of the mounting substrate 340 from passing through the opening 334 to an area below the mounting substrate. With the device 300 in position on the mounting substrate 340, a mounting nut 332 is threaded onto the threaded sink mount 320 until it bears against a lower surface of the mounting substrate 340, thereby securing the device 300 to the mounting substrate 340. The cable bypass channel 328 provides access from below the mounting substrate 340 to one or more connection sockets 330 located on a lower surface of the base 304.

Referring now to FIG. 9, the device 300 is shown mounted to the mounting substrate 340 and connected to a water supply line 144, 146 in a manner similar to that described in previous embodiments. In the pictured embodiment, the tee junction 342 includes a center tap 321 with standard pipe threads for engagement with the threaded water line connector 322, and compression fittings 323 at the ends thereof. The compression fittings 323 enable installation of the device 300 using materials that are readily available, and compatible with hardware commonly used for under-sink water supplies. According to other embodiments, other types of fittings can be used, especially where a monitor device is intended to be connected in other locations or configurations.

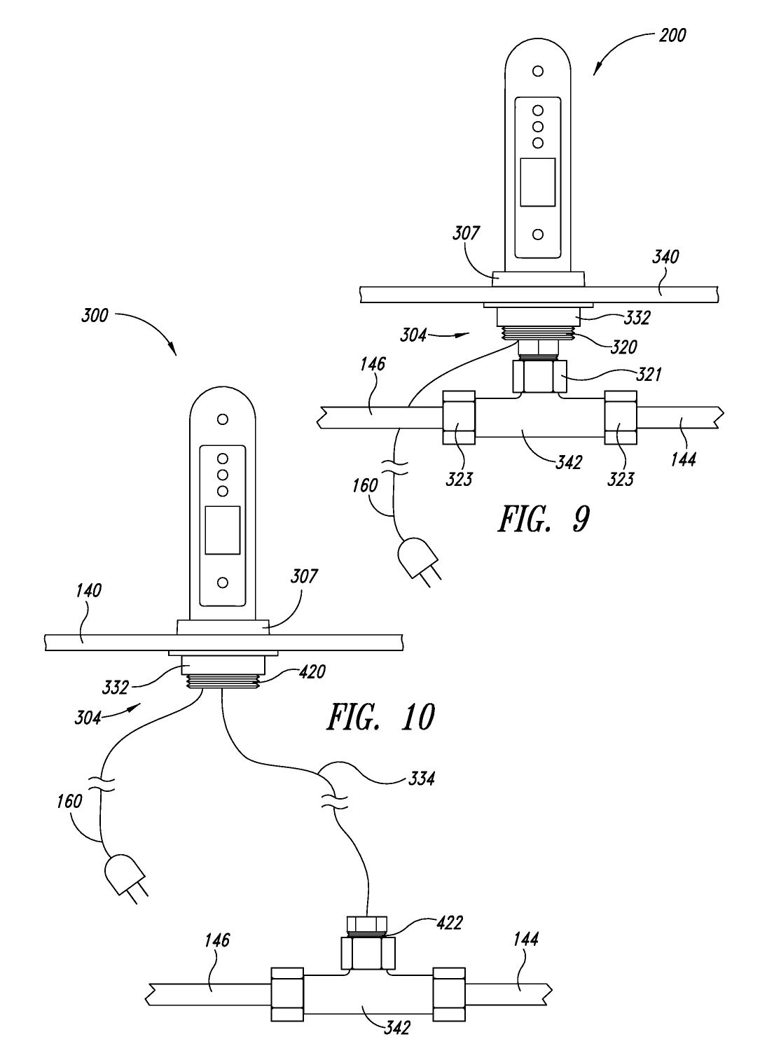

Referring now to FIG. 10, a water quality monitor device 400 is shown according to an embodiment of the invention. The device 400 is shown mounted to a mounting substrate 340 in a manner substantially similar to that described with reference to FIGS. 8 and 9, and the device 400 is functionally similar in many respects to other embodiments previously described. However, the device 400 includes a separate water line connector 422 coupled to the device 400 via a cable 434. Test electrodes are coupled to the water line connector 422 in a manner similar to that described with reference to other embodiments, such that, when the water line connector 422 is coupled to a water line, as shown in FIG. 10, the test electrodes are placed in contact with water passing through the water line, as previously described. The cable 434 extends from the water line connector 422 to the device 400, such that water tests can be conducted, as described with reference to previous embodiments, while the device 400 is mounted some distance from the water line connector 422. The cable 434 may be coupled to the device 400 via a cable bypass channel, as described with reference to previous embodiments, or the cable 434, the power cable 160, a relay cable, etc. can be connected via connection sockets provided in a bottom surface of the threaded sink mount 420 of the device 400.

According to one embodiment, the water line connector, with test electrodes, is configured to be attached to the inlet of a cold-water faucet, and to provide an inlet configured to receive the cold water line. According to another embodiment, the test electrodes are incorporated into the faucet and coupled to a monitor device such as the device 400 shown in FIG. 10. According to another embodiment, a complete water quality monitor device is incorporated into the structure of a faucet.

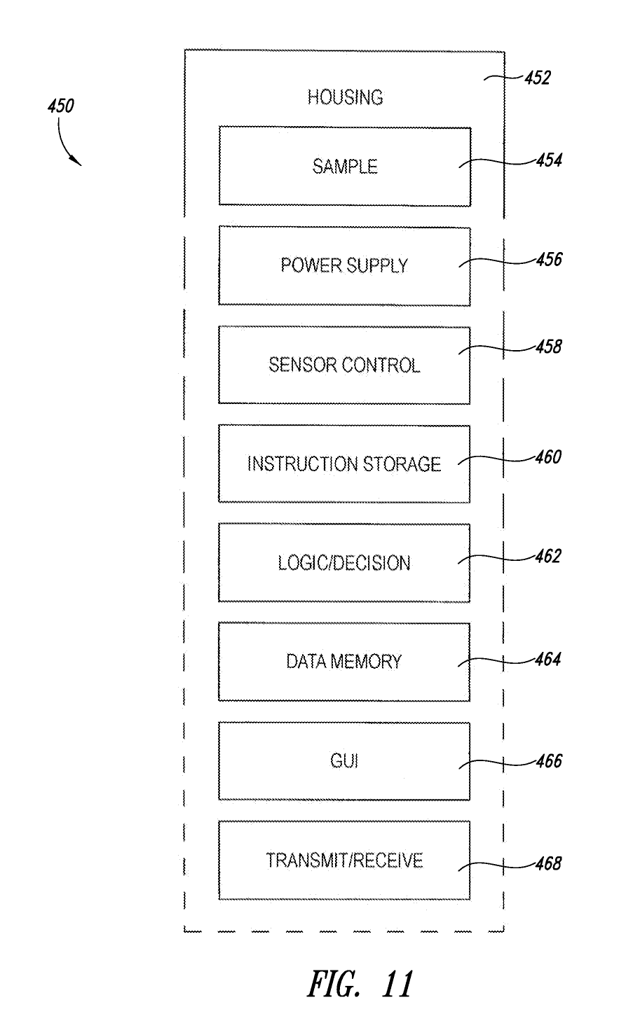

Turning to FIG. 11, a block diagram of a monitor device 450 is illustrated to show components of monitor devices according to various embodiments. The term component is used here primarily in a functional sense to refer to any device or system that operates to perform the given function. The monitor device 450 includes a housing 452, a sampling component 454, a power supply, 456, a sensor controller 458, a instruction storage 460, a logic/decision module 462, a data memory 464, a graphical user interface 466, and a transmit/receive module 468.

The housing 452 is positioned on a fluid transmission line and configured to maintain the sampling component 454 in a position to perform sensing operations on the fluid passing through the transmission line. Accordingly, the housing 452 may be coupled in any of a number of locations that include, for example, the water supply line of a residence or business in the vicinity of the water meter, between the water meter and the first branch of the residence or business, on the cold-water supply of a faucet, at the water intake of a machine, in an underground vault or pipe where treated effluent leaves a treatment plant, at a line where residential effluent leaves a residential septic system, at the outflow point of a rainwater catch basin, etc.

As indicated by the broken lines representing the sensor housing 452, the sampling component 454 is the only component that is location-critical, inasmuch as it must be placed at a location where it can contact the fluid to be monitored. The remaining components of a particular device can be located in the housing 452, stand alone, be located in other housings, or be incorporated into other devices, according to the design requirements of the particular embodiment. For example, FIG. 10 shows an embodiment in which a sampling component, i.e., the test electrodes of the monitor device 400, is positioned in a housing comprising the water line connector 422 and the tee junction 342. All of the remaining components are located in a separate housing mounted to a sink top, and coupled by a cable 434 to the sampling component. In contrast, in the embodiment of FIG. 9, which is otherwise similar to the embodiment of FIG. 10, all of the components of that embodiment are incorporated into a single housing.

The power supply provides the necessary electrical power to operate the sampling component 454 and other components that share a common power connection, and can comprise one or more of a direct connection to a power grid, a battery, a capacitor, a solar collector, a device configured to draw power from a flow of fluid in the transmission line, a charger configured to charge a battery while power from an external source is available, etc.

The sensor controller 458 controls operation of the sampling component 454. The controlled operations can include, for example, controlling the timing and duration of sampling events, production of raw numerical values from the sampling component, converting an analog sensor signal to a digital signal, etc. The sensor controller 458 can comprise a circuit formed on a semiconductor substrate or assembled on a circuit board, instructions stored in a computer-readable medium and executed by a computer processor, etc.

Instruction storage 460 holds values necessary for ongoing operation of the monitor device 450, such as, for example, reference and threshold values, transient values that are held for use in a subsequent operation then discarded, such as previously obtained values for slope determination or recently obtained values for averaging purposes, instructions for handling of data produced, etc. The instruction storage module can comprise electronic buffers or registers, an appropriate number of memory cells incorporated into a circuit with the sensor controller 458, a portion of a larger memory that also includes the data memory 466, etc.

The logic/decision module 462 performs operations that require calculations or manipulation of values that are subject to change, and to indicate appropriate actions. Such operations can include general system monitoring, such as state-of-charge (SOC) of a battery, signaling low SOC, and calculation of sample repetition rate when it depends on factors like fluid flow rate or SOC. The logic/decision module 462 also performs operations that reduce raw data from the sampling component 454 to a form that is useful to a user. These operations can include, for example, averaging, calculating slope values, and comparing sample values with reference or threshold values. The logic/decision module 462 also determines operations to be taken on the basis of the comparisons and other processes. Such operations can include dropping an old value and holding a new value in a moving window average, incrementing a count register, producing an overvalue signal where appropriate, signaling an audible or visual overvalue alarm, signaling a valve controller, etc.

The data memory 464 is a memory configured to save data produced by any of the sampling component 454, the sensor controller 458, or the logic/decision module 462. A data memory module may be employed where historical data is desired, such as to track trends over time, or to compare data collected by one device with that from other devices. The data memory 464 can comprise, for example, a digital memory configured for exclusive recording of data from the device, a memory shared with another device, or a memory associated with a general purpose computer such as a personal computer. The data memory 464 can also comprise a writable storage medium such as a CD-ROM or flash memory.

The GUI 468 provides access to the monitor device 450 by a user, and may comprise, for example, the LCD screen and keypad of a monitor device, a wall-mounted panel, and a handheld remote controller. Additionally, the GUI 468 can comprise components of a typical personal computer such as a screen, keyboard, and mouse.

Finally, the transmit/receive module 468 provides communication between components of the monitor device 450, or between the monitor device and other devices, to send data and to receive operation instructions. According to some embodiments, only a transmitter is provided, such that the device is not capable of receiving. According to embodiments in which components are separate from each other, there may be more than one transmitter or receiver, coupled to a respective ones of the components to permit communication with the other components.

Various embodiments employ different combinations of the components described above. For example, one device provides a sampling component 454, a power supply, a sensor controller, and a transmit/receive module, and only performs an operation when commanded, and immediately transmits data obtained. Other devices collect, process, and save all collected data for future use. Furthermore, components of the monitor device 450 need not be discrete components, but can be integrated with other components on common substrates. In other embodiments, the tasks described above with respect to various components can be distributed differently, such that, for example, one circuit might be configured to perform some of the logic/decision tasks and some of the sensor controller tasks, while a processor of a remote system implements a software program to perform others of the logic/decision tasks and data storage, and so on.

In many cases, the tasks described with reference to the components of the monitor device 450 can be performed by a wide range of devices or combinations of devices that includes, for example, discrete electronic devices assembled into circuits, integrated circuits, dedicated processors, general purpose processors, software programs stored on computer readable media, mechanical devices, etc.

Furthermore, while the functions of embodiments of the invention may be described or claimed with reference to discrete circuits or components, such references are to be construed as including equivalent configurations, including those outlined above. Thus, for example, claims that recite two or more individual circuits, each configured to perform specified tasks, are to be construed as also reading on devices in which the recited tasks are performed by a larger or smaller number of circuits, by circuits performing different combinations of the tasks, or by a single combined circuit, as well as on devices in which some or all of the tasks are performed by more generalized devices such as programmable logic systems and computers.

One issue that may arise in connection with water supplies that have a relatively high level of salts and/or metals in the water is that, over time, the continued application of voltage to the test probes may result in accretion of material, or plating of metals on the negative test probe. In accordance with one embodiment, therefore, the polarity of the voltage applied to the test probes is periodically reversed, in order to reverse the electrolytic effects.

A partial list of water contaminants that can be detected by conductivity testing as described with reference to the principles of the invention includes substances such as aluminum, calcium, chloride, chromium, copper, iron, lead, magnesium, manganese, nitrate nitrogen, sodium, sulfate, and zinc. This list includes many of the most common and most harmful contaminants.

Embodiments of the invention have been described with reference to devices and methods that employ conductivity testing to establish TDS. According to alternate embodiments, characteristics of a water sample other than conductivity may also be employed to test the overall suitability of the water for a selected use. For example, other characteristics that may be monitored include turbidity of water, oxygen content, chlorine content, temperature, pH level, petroleum hydrocarbons, zinc, various heavy metals, etc. Furthermore, according to an embodiment, a monitor device is provided that is configured to monitor more than one water quality characteristic, such as for example, TDS and turbidity, or turbidity, oxygen content, and temperature, etc.

Embodiments of the invention have been described with respect to monitoring potable water with responsive actions such as controlling a valve. According to another embodiment, a monitor device is configured to monitor effluent water and take additional responsive actions, such as, for example, filling a sample vial for testing, or releasing an offsetting agent into the water flow to mitigate an over-limit value. Many septic facilities process incoming sewage to remove solids, chemicals, and microorganisms, then release treated water, generally into drainfields, but also, possibly, other drainage, such as storm water systems, rivers, lakes, etc. The water typically passes through a final sand filter before being released. Allowable residual content of the effluent is generally regulated by local and national agencies, and where regulated limits are exceeded, the owner may be subject to significant fines and/or remediation costs. The inventors recognize that a water quality monitor device configured to monitor TDS, turbidity, or other water quality characteristics can be employed to continually monitor the residual content in the effluent, transmit and store associated data for local and central monitoring, control valves for such things as diverting to backup filtration, and activate alarms as described with reference to other embodiments disclosed. Other applications of such a device include monitoring effluent from factories that use water in fabrication processes, monitoring runoff from highways or parking lots where it flows into municipal drainages, monitoring storm water draining from localized storage and sediment facilities for storm water treatment, monitoring water released from cooling systems of power plants and factories, and monitoring outflow from septic systems of individual residences.

In some cases, water will not always be present in a system, or may not always be flowing. This is the case, for example, in storm water catch basins, retention ponds, vaults, etc., all of which are referred to hereafter generically as basins. Some are configured to catch and hold storm runoff water and allow it to percolate back into the ground water, while others hold runoff water and allow it to flow at a controlled rate into a larger drainage system. Such basins may be dry for part of a typical year, but more or less full of water during others. Such basins may also be provided with overflow systems to permit water that exceeds some level to flow into other drainage systems. Regulations that govern such systems vary according to a system's location, size, configuration, age, etc. Many basins are configured to filter or treat runoff water to remove contaminants and pollutants. In some cases, regulating agencies require the submission of regular reports by the property owner, operator, or tenant (hereafter, generically, owner) of a basin, detailing water levels and effluent flow, as well as the levels of various contaminants.

For example, in a case known to the inventors, a Federal agency requires quarterly reports showing results of regularly performed tests on released water for TDS, TSS, turbidity, and zinc. However, the requirement does not provide information on how to conduct the tests, and there is insufficient funding for regular monitoring for compliance or accuracy. As a result, there is potential for falsification of the reports, and, even where owners make genuine efforts to comply with the requirements, there is little confidence in the resulting data.

Total suspended solids, in particular, is almost always a concern with surface runoff water. TSS tests are labor intensive and time consuming. While suspended solids in water are the primary cause of turbidity, and turbidity can be measured relatively easily, it cannot be used to provide an accurate value of TSS without first establishing a correlation, because different kinds of particlates have unequal effects on turibidity, for a given level of TSS. However, if such a correlation is made, thereafter turbidity can be used to provide a general indication of TSS at a given location. Typically, the overall composition of suspended solids at a given site will remain fairly consistent, with concentrations of particular contaminants varying proportionately over time.

According to an embodiment, a TSS measurement is performed at long intervals to establish and track the correlation with turbidity. A device is then configured to perform regular turbidity tests and provide data from which approximate values of TSS are derived on the basis of the previously established correlation. In this way, the frequency of the much more costly and time consuming TSS tests is greatly reduced, while first order tracking is provided using an inexpensive automated turbidity test.

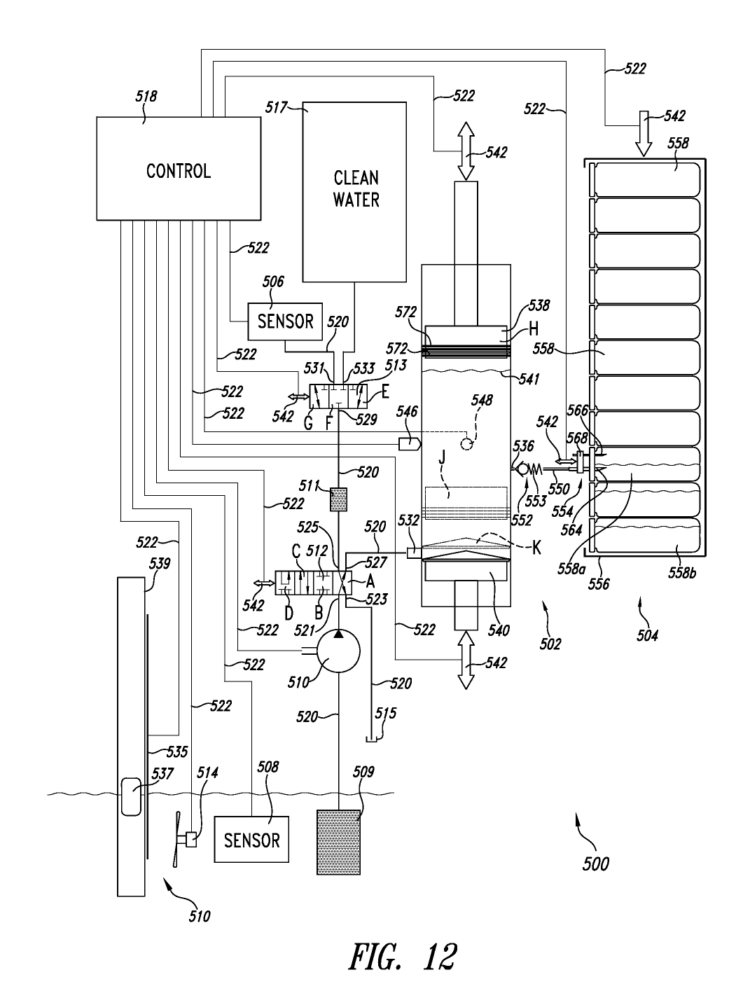

FIG. 12 illustrates, in diagrammatic and schematic form, a monitor device 500 according to an embodiment, provided for use in monitoring water 501 in a basin. The device 500 includes a water sampler device 502, a vial handling mechanism 504, sensors 506, 508, a fluid pump 510, first and second switching valves 512, 513, a flow detector 514, a water level detector 516, a clean water tank 517, and a control unit 518. Fluid transmission lines 520 provide fluid coupling, and control cables 522 are provided, by which the control unit 518 controls system operations and processes, and receives data from sensors. Actuators for changing positions of various elements are indicated generally at 542.

The fluid pump 510 is coupled to draw water from the basin through a coarse filter 509. The filter 509 is sized to pass any particles that are likely to remain in suspension, but to remove larger particles that might otherwise tend to foul the system or interfere with its operation. The first switching valve 512 is a four way four position valve, with a first valve port 521 coupled to an outlet of the pump 510, a second valve port 523 coupled to a sump 515 (which may be nothing more than a drain back into the basin) a third valve port 525 coupled to the second switching valve 513, and a fourth valve port 527 coupled to the water sample device 502. In the first valve position A, the first valve port 521 is placed in fluid communication with the fourth valve port 527 while the second valve port 525 is coupled to the third valve port 523; with the first valve 512 in the second position B, all valve ports are closed; in the third position C, the first and third valve ports 521, 525 are coupled and the second and fourth valve ports 523, 527 are coupled; and in the fourth valve position D, the third and fourth valve ports 525, 527 are placed in fluid communication, while the first and second valve ports 521, 523 are closed.

The second switching valve 513 is a three way, three position valve, with a first valve port 529 coupled to the third valve port 525 of the first valve 512 via a fine filter 511, a second valve port 531 coupled to the sensor 506, and a third valve port 533 coupled to the clean water tank 517. When the second valve 513 is in its first position E, its first valve port 529 is placed in fluid communication with its second valve port 531 and the third valve port 533 is closed; in the second position F, all ports of the second valve 513 are closed; and in the third position G, the first and second valve ports 529, 531 are placed in fluid communication.

The flow detector 514 is configured to detect water flow and speed, and the water level detector 516 includes a sensor 535 configured to detect the position of a float 537 within an open cylinder 539. Thus, according to an embodiment, the system 500 can be configured to perform tests or obtain samples in response to significant changes in flow rate, water level, and when water flows in an overflow channel, and can be configured to suspend operation when the water level drops below a selected point.

The water sampler device 502 is configured to draw a physical water sample for lab analysis, and also, in the embodiment shown, to measure turbidity. The device 502 comprises a hollow cylinder 530 made from a transparent material such as glass or Pyrex®. The cylinder 530 includes a supply port 532 and a sample port 536. First and second pistons 538, 540 are positioned within the cylinder 530, with respective actuators 542 coupled to control movement of the pistons within the cylinder. A light emitter 546 and detector 548—shown in dotted lines to indicate a position behind the cylinder 530—are coupled to an outer surface of the cylinder. A sample outlet tube 550 is coupled to the cylinder 530 at the sample port 536, and includes a check valve 552 biased in a closed position by a spring 553. A sample dispenser mechanism 554 is coupled to the sample outlet tube 550, as will be described in more detail later.

The first piston 538 comprises a plurality of annular sealing members 572, formed of a resilient material such as, for example, natural or synthetic rubber, in sealing engagement with an inner surface of the cylinder 530. The first piston 538 is configured to move within the cylinder 530 between a first position H and a second position J. The second piston 540 comprises at least one annular sealing member 574 in sealing engagement with the inner surface of the cylinder 530. The second piston 540 is configured to move between a first position K and a second position L.

The vial handling mechanism 504 comprises a vial magazine 556 loaded with a plurality of sample vials 558. In the embodiment shown, the vials 558 are stacked on their sides vertically in the magazine 556. Each vial 558 is sealed with a crimped-on cap 560 that includes a natural or synthetic rubber cover and seal, such as are well known in the medical arts for the dispensing of medicaments via hypodermic needles. The vial magazine 556 is translatably positioned within a carrier that is movable vertically to position each vial, in sequence, opposite the sample dispenser mechanism 554. Details of the carrier are not shown, and will vary according to the embodiment. However, it is well within the abilities of one of ordinary skill to provide the elements of the vial handling mechanism 504 without undue experimentation.

The sample dispenser mechanism 554 includes a needle block 568 that carries a sample needle 564 that slides or telescopes on the sample outlet tube 550 and a vent needle 556. The needle block 568 moves horizontally with the sample and vent needles 564, 566. When a water sample is to be obtained, the needle block 568 is extended with the sample and vent needles 564, 566 until they each pierce the seal of the vial 558 positioned opposite, and the sample of water is dispensed into the vial via the sample needle 564, while air in the vial is vented via the vent needle 566. When the water sample has been dispensed, the needle block 568 is withdrawn from the vial 558. Once the needles are withdrawn, the magazine 556 is moved downward until the next vial 558 is positioned to receive a sample. According to an alternative embodiment, the vials are vacuum charged such that, when the sample needle 564 pierces the seal, the water sample is drawn by the vacuum into the vial 558. With such an embodiment, a vent needle is not required. According to an alternative embodiment, a tamper evident seal is applied over the cap.

While, as discussed below, the system is configured to flush the sample needle 564, after each sample procedure, this may not be sufficient to prevent some residue accretion in the needle. Accordingly, the sample needle 564, or the needle block 568, may be regarded as a consumable that the user will replace each time the vial magazine 556 is reloaded. Other elements of the system 500 may likewise be replaced regularly to prevent buildup of matter that might otherwise affect the accuracy or functionality of the system.

The magazine 556 is easily removable for service and refill. The user can either pull the magazine and reload it on site, or can bring a loaded magazine 556 and simply exchange it with the magazine in the system. According to one embodiment, each vial is provided with a unique machine-readable label, such as a bar code, that is read into a memory in the control unit 518 as the sample is drawn, and data associated with the sample in each vial are also saved to the memory. Such data may include, for example, time, date, turbidity, turgidity, water temperature, water level in the basin, volume and speed of flow, etc. Correlation of such data taken concurrently with the drawing of each physical sample may be of significant value not only in tracking fluctuations of various characteristics, but also in more accurately interpreting the analysis of the sample, and in calibrating other sensors of the system. According to an alternate embodiment, the unique identifying code of each vial is entered into the memory in sequence, either automatically or manually by the user, when the magazine is loaded into the system.

The vial handling mechanism 504 can be configured to retain all the vials 558 in the magazine 556, with the user removing the filled vials prior to reloading the magazine. According to another embodiment, the mechanism 504 is configured to dispense the filled vials to an outlet chute or port of the system, where they can be retrieved as they are filled. The latter embodiment permits the analysis of the water in the vials on a more nearly real-time basis. The system 500 can be configured to collect physical water samples in response to one or more of a number of conditions, including, for example, regularly timed intervals, when one or more of the other sensors associated with the system 500 detects an overvalue of a water quality characteristic relative to a selected threshold, when the water level or rate of flow in the basin rises or drops to a selected level; when the user signals for a sample, and according to any criteria that are deemed significant by the user.

Operation of the water sampler device 504 is described hereafter. According to the embodiment shown, the various steps of the process are controlled by the controller 518 via actuators 542. However, as with other disclosed embodiments, the scope of the invention is not limited to the specific configuration shown. With the cylinder 530 empty, the first piston 538 is positioned at its second position J and the second piston 540 is positioned at its second position L. The valve 512 is placed in its first position A placing the pump 510 in fluid communication with the cylinder port 532. The pump 510 is controlled to draw water from the basin via the filter 509, and pump the water into the cylinder 530 via the port 532. As water enters the cylinder 530, the first piston 538 moves upward at a rate that approximately corresponds to the fill rate of the water in the cylinder 530. When the water in the cylinder reaches a selected level, the pump 510 stops operation and the second piston 540 moves upward to its first position K, thereby closing the inlet port 532 and capturing a column of water 541 within the cylinder 530. According to an alternate embodiment, the port 532 of the cylinder 530 is coupled via the filter 509 to the water 501 in the basin. Vacuum generated by upward movement of the first piston 538 is sufficient to draw water into the cylinder via the filter 509.

The system waits for a selected period of time during which matter that is present in the column of water 541 is permitted to settle to the bottom or float to the top of the water in the cylinder 530. The period is selected to be sufficiently long to permit most or substantially all of the non-suspended solids to settle from the water, and may be, for example, one hour or several hours. At the end of the wait period, the system proceeds. If a turbidity test is to be performed, the light emitter 546 is controlled to emit light into the cylinder 530, and the detector 548 detects a portion of the light that is scattered by suspended particles in the column of water. A turbidity value can be derived from the intensity of the scattered light relative to the intensity of the emitted light. The emitter 546 and detector 548 are positioned in a plane about midway between the top and bottom of the water in the cylinder 530 so as to obtain a measurement that, to the extent possible, is influenced only by suspended solids. The embodiment of FIG. 12 is shown and described as having a single emitter 546 and a single detector 548 oriented at right angles with respect to each other. According to other embodiments, additional emitters and detectors are employed in configurations that are well known in the art, including an embodiment in which two emitters and two detectors are employed, with each emitter being positioned directly opposite from a respective detector.

If a physical water sample is to be obtained, the first piston 538 moves downward in the cylinder 530. The spring 553 of the check valve 552 has a bias force selected to hold the valve closed when the cylinder 530 is full of water. However, when the first piston 538 moves downward in the cylinder 530 while the second piston 540 remains in its first position K, water pressure in the cylinder 530 increases until the bias force of the spring 553 is overcome, and the check valve 552 opens, allowing water to flow from the sample port 536. Preferably, a small amount of water is driven through the sample port 552 before the actual sample is collected, to flush the sample port 536 of any residue from previous water samples. The actuator 570 is then controlled to move the needle block 568 outward to pierce the seal of a vial 558 and dispense the water sample, as described above.

In order to obtain a water sample that is substantially free of non-suspended solids, the volume of the cylinder 530 is selected to be significantly greater than the volume of a sample vial 558, and the sample port 536 is positioned well above the bottom of the water column in the cylinder 530. Thus, water from the clearest portion of the water column is dispensed into the sample vial 558.

When the system has completed the test and sample procedures, the first valve 512 moves to its third position C, placing the port 532 in fluid communication with the sump, and the second piston 540 moves to its second position L, which again opens the port 532. The first piston 538 moves downward toward its second position J, driving water from the cylinder 530 via the port 532. While only one port 532 is shown in FIG. 12, a plurality of ports can be provided, distributed around the circumference of the cylinder 530. Additionally, an upper surface 541 of the second piston 540 can be shaped to cooperate with the one or more drain ports 534 to generate water movement in the cylinder 530, as it is draining, that tends to scour sediment deposited on the upper surface 541 during the wait cycle. The first piston can also be driven downward with increased speed and force to increase the cleaning effect.

The plurality of annular sealing members 572 of the first piston 538 thoroughly wipe the inner surface of the cylinder 530 during each filling and draining cycle. This serves to keep the inner surface of the cylinder 530 substantially clean, and free of deposits that might otherwise attenuate light transmitted by the emitter 546 and detected by the detector 548, and that would otherwise interfere with an accurate turbidity reading. In embodiments where an emitter is positioned directly opposite a detector, on opposite sides of the cylinder 530, the system can be configured to perform a turbidity-type test through the empty cylinder 530 just prior to filling the cylinder for a turbidity test. In this way, the system can compensate for any deposits on the cylinder that are not removed as the piston 438 passes, and that would otherwise affect the accuracy of the test.

According to an embodiment, the cylinder 530 is flushed with clean water following a test/sample cycle. The second valve 513 is moved to its first position E and the first valve 512 is moved to its fourth position D, placing the clean water tank 517 in fluid communication with the cylinder port 532. The first piston 538 then moves from its second position J to its first position H, drawing water from the clean water tank 517 into the cylinder 530. The second piston 540 moves from its second position L to its first position J, and the first piston 538 moves downward again, increasing the pressure until the check valve 552 opens to flush the sample tube with fresh water. The second valve 512 then moves to its third position C and the second piston 540 moves to its second position L, opening the port 532. The first piston moves downward toward its second position J, flushing the water from the cylinder 530.

It may be necessary or desirable to perform tests on water from which the suspended solids have been removed. For example, the suspended solids might affect the accuracy of a conductivity test performed to determine TDS. As noted above, according to one definition, 0.45 microns is the threshold separating suspended solids from dissolved solids. Accordingly, the fine filter 511 is provided, configured to trap particles larger than 0.45 microns. The first valve 512 is moved to its third position C and the second valve is moved to its third position G. The pump 510 is operated to draw water from the basin via the coarse filter 509 and pump the water to the sensor 506 via the fine filter 511. The sensor 506 is provided with a reservoir to receive and hold the water long enough to perform the prescribed test. When sufficient water has been pumped to the sensor 506, the second valve moves to its second position F and the pump 510 turns off. When the test is complete, the first valve moves to its first position A and the second valve moves to its third position C. This places the sensor 506 in fluid communication with the sump, via the fine filter 511. As the water flows from the sensor back through the filter 511 in the reverse direction, much of the previously trapped particulate matter is flushed from the filter, extending its useful life. Water from the clean water tank 517 can also be used to flush the filter 511, as well as the filter 509. The filter 511 should also be flushed prior to rinsing the cylinder 530 of the water sampler device 502 to avoid carrying contaminants from the filter 511 into the cylinder 530. According to alternative embodiments, additional valving and fluids lines are provided to shunt clean water around the filter 511 for rinsing the cylinder 530, and for rinsing or flushing other components of the system.

According to an embodiment, the clean water tank 517 is replenished by the pump 510 via the first and second valves 512, 513 and the coarse and fine filters 509, 511. According to another embodiment, the clean water tank 517 is refilled during periodic servicing. According to a third embodiment, the system is plumbed to a source of clean water such a municipal water supply, which is used either to maintain the water level in the clean water tank 517, or to eliminate the tank entirely.

The sensor 508 is positioned and configured to monitor or sample characteristics of the water continually or as it is found in the basin. Like the sensor 506, other sensors are configured to be in contact with the water only while in operation, and may monitor characteristics of the water less frequently. The sensors 506 and 508 are shown generically, and the system is not limited to the two sensors shown, but can include any number of sensors or sample devices. The sensors can be configured to monitor any water quality characteristic required, many of which have been listed above. Additionally, the sensors can be configured and positioned to monitor water characteristics at specific locations, such as, for example, the point of inflow or outflow of a basin, a point near the bottom of the basin or near the surface of the water in the basin, etc. Furthermore, the system 500 can be configured, by placement of sensors or intake lines, to sample same characteristics at various points in the basin. For example, it may be desirable to monitor TDS of water as it flows into the basin as well as when it flows out, in which case, TDS sensors can be positioned at both locations.

The control unit 518 includes components necessary to control the operation of the system 500, which can include for example, many or all of the components and configurations detailed above with respect to FIG. 11. According to an embodiment, the control unit 518 is modular, and configured such that many components can be added or removed without affecting the functionality of the system 500. In particular, a plurality of connection ports are provided, to which the control cables 522 of the sensors and actuators of the system are coupled. Each such element includes an instruction set for its own operation, such that when a new sensor or other device is added to the system 500, the control unit 518 can immediately control the operation of that device, and record data collected, without requiring additional programming or hardware. The user merely connects the device and enters any necessary operating parameters, such as, for example, frequency of operation, threshold levels, etc. The instruction set can be in the form of software that is uploaded to the control unit 518 or that is read and executed by the component itself, or can be in the form of hardware, or firmware, or a combination of the above. Thus, a system 500 can be configured to meet the testing requirements of many different effluent sources and water bodies, such as collection and retention basins, drainage and irrigation ditches, ponds, canals, rivers, lakes, water treatment plants, etc., and also to meet the changing requirements of particular sources as conditions and concerns change.

As noted above, many local and federal regulations require property owners or tenants, as a condition of development or land use, to provide and maintain some kind of runoff collection system, to take measures to ensure that pollutants do not enter the groundwater or drainage from the runoff, and to report on specific characteristics of the water in the system at regular intervals.

According to an embodiment, the system 500 is partially or wholly encased in a tamper-resistant housing to prevent modification or interference by unauthorized parties. The system is initially installed by a regulating government agency or a licensed contractor. This can be at the agency's expense, or at the property owner's expense, where the owner is required to monitor and report water quality. Access to the system is thereafter limited to the government agency or contractor to ensure honest and accurate monitoring and reporting. When the system 500 is first installed, a TSS measurement is obtained by known methods. The sample dispenser mechanism 554 is cycled through a turbidity test, and the results of both tests are processed to produce a correlation of TSS and turbidity at that site. Thereafter, provided it is possible, given the particular purpose for which each physical water sample is originally drawn, some or all of the samples are also analyzed for TSS, and the resulting values is compared to a value predicted by the turbidity test performed on the water used to fill the vial. The correlation between turbidity and TSS for that particular site is then updated.

The system 500 is configured to transmit collected data to the agency, either as it is collected, or at regular intervals. This allows the agency to collect and correlate the data from a large number of sources without frequent onsite visits and at increased accuracy and reliability. More widely spaced visits to collect physical samples and service the systems can be performed more quickly and with less effort. Resources previously spent manually taking samples and performing tests at limited numbers of problem sites can be spent collecting samples, refilling vial magazines, and otherwise servicing monitor devices of larger numbers of sites. Additionally, it may be determined that at some sites, physical water samples are not required, or are required at a much reduced frequency, which means that service calls to these sites can be at very widely spaced intervals. This, in turn, permits the regular collection of reliable data from many more sites without overextending resources, which provides a broader and deeper picture of the surface water conditions of a given region. Finally, in many cases, especially after levels of trust and confidence are established, property owners or tenants may be relied upon to perform the necessary service, and to forward the sample vials as they are filled.

In addition to transmitting data, the system 500 can be configured to run self-test routines and transmit diagnostic information. For example, the system can signal when the vial magazine 556 is empty or nearly so; the condition of the filters 509, 511 can be monitored by the system by tracking the power expended by the pump 510 while drawing water through the filters; the system can signal when its housing has been opened; if the system runs on a battery, the system can indicate a low battery or a problem with a charging system; etc.

The system can also be configured to send the data to the property owner, allowing the owner to review the same data. This gives the owner the opportunity to quickly address problems that could otherwise develop into costly problems, either because of fines associated with water contamination or with the expense of clean up. For example, high levels of zinc may indicate that rainwater is leaching zinc from galvanized roofs on the property, which may be eliminated by applying a protective coating to the metal. However, if it continues for an extended period, the owner may be subject to fines and the cost of removing contaminated soil. Petroleum hydrocarbons are particularly common contaminants, which can be filtered from runoff water at the outlet of a basin or vault, but can require significant remediation if allowed to percolate into groundwater. On the other hand, if the owner believes the data is inaccurate, the owner can conduct independent tests and compare them with the system's results taken at the same time and location.

The system can be prompted, or “pinged” to collect a physical water sample by the agency or by the property owner. The system can also be pinged to collect two samples, one after the other. In this way, if the owner or agency wishes to definitively confirm the accuracy of the system and the analysis procedure, one vial can be processed through the regular channels while the other is sent to an independent lab for comparison processing. By giving the property owner the ability to signal for a physical sample, a check is provided in the process to keep the regulating agency accountable for its operation of the system.

The system 500 has been described for extended, long term use with reference to installations such as catch basins and vaults, but the system can also be used in temporary installations. For example, construction sites are often sources of contamination of runoff water because of the materials and machinery commonly used. Where a builder is required to monitor runoff from such a site, the system 500 can be installed in a vault or housing at the lowest point of the site to monitor the runoff until the construction project is completed or there is no longer any need for the system.

For the purposes of the claims, when used with reference to an installed system, the term permanent is used to refer to a system that is configured to remain where installed for longer than the time necessary to perform one or two tests, is configured to operate without direct human supervision or observation, and that includes operating instructions by which it can determine actions to take and when to take them, without requiring ongoing human interaction. The term does not refer to devices, for example, that are emplaced to perform a small number of specific actions before being moved to another location for similar actions, such as would be the case where an individual(s) is transporting a testing device to measure one or more water characteristics at a plurality of sites, sequentially.

Ordinal numbers, e.g., first, second, third, etc., are used in the claims merely for the purpose of clearly distinguishing between claimed elements or features thereof. The use of such numbers does not suggest any other relationship, e.g., order of operation or relative position of such elements. Furthermore, ordinal numbers used in the claims have no specific correspondence to those used in the specification to refer to elements of disclosed embodiments on which those claims may read.

The abstract of the present disclosure is provided as a brief outline of some of the principles of the invention according to one embodiment, and is not intended as a complete or definitive description of any embodiment thereof, nor should it be relied upon to define terms used in the specification or claims. The abstract does not limit the scope of the claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. In particular, the summary and abstract are intended to provide brief outlines of embodiments of the invention, and are not to be considered complete or limiting with respect to any aspect of the invention. The invention is not limited except as by the appended claims.

1. A fluid quality monitor device, comprising:
first and second electrodes;
a first housing configured to be coupled in series in a fluid transmission line, the first and second electrodes being positioned in the housing such that a portion of fluid passing through the transmission line passes between the first and second electrodes;
a power circuit configured to repeatedly apply a voltage potential across the first and second electrodes;
a measurement circuit configured to produce a current flow measurement correlated to current between the electrodes during each application by the power circuit; and
a comparison circuit configured to compare a reference value with succeeding current values, each derived from one or more of the current flow measurements, and to produce an overvalue signal if a latest current value exceeds the reference value.

2. The device of claim 1, comprising an alarm circuit configured to produce an overvalue alarm if the comparison circuit produces an overvalue signal.

3. The device of claim 1, comprising a calculation circuit configured to derive each current value from an average value of a respective plurality of current flow measurements.

4. The device of claim 3 wherein the comparison circuit is configured to derive the reference value from an average value of a first obtained plurality of current flow measurements.

5. The device of claim 1 wherein the comparison circuit is configured to compare the latest current value with a previously derived current value and to produce an overvalue signal if a difference between the latest current value and the previously derived current value exceeds a slope threshold.

6. The device of claim 1 wherein the comparison circuit is configured to compare each current value with the reference value, and to produce an overvalue signal only if:
the latest current value exceeds the reference value, and
a difference between a previously derived current value and the reference value is less than a maximum slope threshold.

7. The device of claim 6 wherein the comparison circuit is configured to compare the latest current value with a previously derived current value and:
if the latest current value is lower than the previously derived current value or exceeds the previously derived current value by less than the maximum slope threshold, then holding the latest current value for comparison as the previously derived current value in a next succeeding operation of the comparison circuit, and
if the latest current value exceeds the previously derived current value by more than the maximum slope threshold, then holding, for comparison as the previously derived current value in a next succeeding operation of the comparison circuit, a value equal to a sum of the previously derived current value and the maximum slope threshold value.

8. The device of claim 1, comprising a second housing, and wherein the calculation, comparison, and alarm circuits are mounted in the second housing.

9. The device of claim 8, comprising a transmission circuit mounted in the first housing and configured to transmit current flow measurements to the calculation circuit in the second housing.

10. The device of claim 1, comprising a fluid valve having a control input coupled to the alarm circuit and configured to close when the alarm circuit produces an overvalue signal.

11. The device of claim 1, comprising a memory configured to sequentially save derived current values.

12. The device of claim 11 wherein the memory is configured to store the reference value.

13. The device of claim 1, comprising a transmission circuit configured to transmit current values to a remote receiver.

14. The device of claim 1, comprising receiver circuit configured to receive the reference value from a remote source.

15. A central water quality monitor system, comprising:
a first water quality (potability, purity) sensor device [global] coupled to a first water supply line of a first water consumer and configured to obtain an instantaneous value of a water quality characteristic of water in the first water supply line and transmit data related to the water quality value of water in the first water supply line;
a second water quality monitor coupled a second water supply line of a second water consumer and configured to obtain a water quality value of water in the second water supply line and transmit data related to the water quality value of water in the second water supply line; and
a data collection facility configured to receive and compile the transmitted data from the first and second water quality monitors.

16. The water quality monitor system of claim 15 wherein the first water consumer is a residence, and the first water quality monitor is configured to transmit the data related to the water quality value of water in the first water supply line to a personal computer at the residence, and the personal computer at the residence is configured to transmit the data to the data collection facility.

17. The water quality monitor system of claim 15 wherein the first water quality monitor is configured to store data related to the water quality value of water in the first water supply line for periodic transmission to the data collection facility.

18. The water quality monitor system of claim 15, further comprising:
a plurality of water quality monitors in addition to the first and second water quality monitors, each of the plurality of water quality monitors being coupled to a water supply line of a respective water consumer [broaden beyond homeowner] and configured to obtain a water quality value of water in the water supply line of the respective water consumer and transmit data related to the water quality value of water in the water supply line of the respective water consumer to the data collection facility.

19. The water quality monitor system of claim 15 wherein the first water quality monitor is configured to periodically obtain a new water quality value of water in the first water supply line.

20. The water quality monitor system of claim 19 wherein the first water quality monitor is configured to derive each water quality value from data collected from a selected number of measurements of a characteristic of water in the first water supply line.

21. The water quality monitor system of claim 19 wherein the first water quality monitor comprises a monitor component and a memory component, and wherein the memory component is configured to receive and store data from the monitor component related to water quality in the first water supply line and to transmit stored data to the data collection facility.

22. The water quality monitor system of claim 15 wherein the first water quality monitor is positioned to obtain the water quality value of water coming into the first water supply line from a water main.

23. The water quality monitor system of claim 15, comprising a water control valve, wherein the first water quality monitor is configured to provide a control signal to the control valve.

24. A water quality monitor system, comprising:
a sensor configured to periodically obtain water quality readings from water flowing in a water supply line;
a memory configured to store the water quality readings from the sensor, together with a time-stamp associated with each reading and indicating the time at which the respective reading was obtained; and
a transmitter coupled to the sensor and configured to transmit the water quality readings.

25. The system of claim 24 wherein the memory is coupled to the sensor and configured to store water quality readings as they are obtained by the sensor, and to provide batches of readings and time-stamps to the transmitter for periodic transmission.

26. The system of claim 24 wherein the memory is coupled to a receiver and configured to store water quality readings as they are transmitted by the transmitter.

27. The system of claim 26 wherein the memory is configured to store water quality readings as they are transmitted by a plurality of transmitters.

28. The system of claim 24 wherein the sensor is configured to obtain a water quality reading upon receipt of a read-command signal.

29. The system of claim 24 wherein the sensor is configured to obtain water quality readings at selected time intervals.

30. The system of claim 24 wherein the sensor is configured to obtain water quality readings at a rate that is directly related to a volume of water flowing in the water supply line.

31. The system of claim 24 wherein the transmitter is configured to transmit an identifier unique to the sensor.

32. The system of claim 24 wherein the transmitter is configured to transmit at selected time intervals.

33. The system of claim 24 wherein the transmitter is configured to transmit upon receipt of a ping from a remote receiver.

34. The system of claim 24 wherein the transmitter is configured to provide a read-command signal to the sensor upon receipt of a ping from a remote receiver.

35. The system of claim 24, comprising a remote relay device configured to receive data from a plurality of transmitters and relay the data to a central collection facility.

36. The system of claim 35 wherein the remote relay device is configured to collect and store data received and periodically transmit collected data to the central collection facility.

37. A method for monitoring surface runoff water, comprising:
obtaining a value of total dissolved solids of water in a basin;
obtaining a first turbidity value of the water in the basin from a device installed permanently at the basin;
deriving a correlation between the value of total suspended solids and the turbidity value; and
on the basis of the derived correlation, estimating a value of total suspended solids of water in the basin from each of a plurality of turbidity values automatically obtained at selected intervals by the device.
